(19) United States
(12) Patent Application Publication
(10) Pub. No.: US 2025/0160883 A1
(43) Pub. Date: May 22, 2025

(54) TARGET DEVICE FOR THE TARGETED GUIDANCE OF A HOLLOW NEEDLE DURING AN ENDOSCOPIC SURGICAL PROCEDURE

(71) Applicant: Richard Wolf GmbH, Knittlingen (DE)

(72) Inventors: Sebastian FREY, Waghäusel (DE); Silas MAUL, Pforzheim (DE); Dirk GÖTHEL, Kürnbach (DE); Frederick POTHOF, Bruchsal (DE)

(21) Appl. No.: 18/841,464

(22) PCT Filed: Feb. 23, 2023
(86) PCT No.: PCT/DE2023/200037

§ 371 Date: Aug. 26, 2024

(30) Foreign Application Priority Data
Mar. 14, 2022 (DE) 10 2022 202 486.9

(51) Int. Cl. A61B 17/34; A61B 1/00; A61B 1/317; A61B 17/00

(52) U.S. Cl. CPC A61B 17/3403; A61B 1/00154; A61B 1/317; A61B 2017/00296; A61B 2017/00477; A61B 2017/00526; A61B 2017/3411

(57) ABSTRACT

A targeting device (9) for the targeted guidance of a hollow needle (17) with an endoscopic-surgical operation. The targeting device (9) is fastened or can be fastened to an endoscopy unit (1) which extends essentially in an endoscopy longitudinal axis (L) and defines a viewing region (11). The targeting device (9) defines a plurality of hollow needle guides (21). Each of the hollow needle guides (21) each extends towards the viewing region (11) along a different polar angle (θ) with respect to the endoscope longitudinal axis (L). Each of the hollow needle guides (21) is open towards one of its two azimuthal sides over its entire length.

### CROSS REFERENCE TO RELATED APPLICATIONS

This application is a United States National Phase Application of International Application PCT/DE2023/200037, filed Feb. 23, 2023, and claims the benefit of priority under 35 U.S.C. § 119 of German Application 10 2022 202 486.9, filed Mar. 14, 2022, the entire contents of which are incorporated herein by reference.

### TECHNICAL FIELD

The present invention relates to a targeting device for the targeted guidance of a hollow needle with an endoscopic surgical operation, as well as to an endoscopy system with such a targeting device, in particular for minimal-invasive surgery on the spinal column.

### BACKGROUND

There are already endoscopy systems for minimal-invasive endoscopy on the spinal column. EP 2 135 542 B1 describes for example an endoscopy unit which is envisaged for minimal-invasive endoscopic spinal column endoscopy, in the form of an endoscope and shank system with an access tube and an endoscope which can be inserted into the access tube. Typically, decompressions of the cervical, thoracic or lumbar spine are carried out herewith, for example in the case of slipped discs, spinal cysts or spinal column stenoses. Herein, the endoscopy longitudinal axis can run for example transforaminally, extraforaminally or interlaminarily.

A problem of the known solution is that the access shank only provides a limited amount of space and provides an access between the vertebral bodies from only one direction. However, for certain surgical operations, it is however advantageous or indeed necessary to provide a second access into the intervertebral space from another direction, additionally to the access into the intervertebral space which is created by the endoscopy unit.

Herein, the second access can be pierced from a different angle by way of a hollow needle, in order to place a guide wire which is located therein. After the placement of the guide wire has been effected, the hollow needle can be withdrawn proximally from the guide wire and a rigid or flexible working sleeve can be pressed in distally via the guide wire. Several working sleeves can also be successively pushed over one another, in order to achieve a necessary access cross section. As soon as the required access cross section is reached, the guide wire as well as possibly inner working sleeves can be proximally pulled out of the outermost working sleeve.

However, the correct alignment and placing of the hollow needle in order to precisely hit the desired target region is problematic. Until now, this has been carried out by very experienced surgically trained personnel amid x-ray and/or ultrasound control. However, there is always a risk of the piercing of the hollow needle not being optimally placed on the first attempt and possibly having to be placed afresh in one or more further attempts.

### SUMMARY

It is an object of the invention to simplify the piercing of the hollow needle and thus to reduce the risk of incorrect piercings.

According to a first aspect of the present disclosure, a targeting device for the targeted guidance of a hollow needle of a certain length with an endoscopic-surgical operation is provided for solving this problem, wherein the targeting device is fastened or can be fastened to an endoscopy unit which extends essentially in an endoscopy longitudinal axis and defines a viewing region, wherein the targeting device defines a plurality of hollow needle guides, wherein each of the hollow needle guides each extends towards the viewing region along a different polar angle with respect to the endoscope longitudinal axis, wherein each of the hollow needle guides is open towards one of its two azimuthal sides over its entire length.

The viewing region can be defined as a focal plane in the object space which lies distally in front of the endoscopy unit and which can be observed by way of an endoscope of the endoscopy unit. For understanding the present disclosure, it is suitable to define a local spherical co-ordinate system of the endoscopy system, consisting of the endoscopy unit and the targeting device which is assembled thereon. The center of the spherical coordinate system can herein be defined as the center of the viewing region which is arranged distally in front of the endoscopy unit. The endoscopy longitudinal axis can accordingly be defined as a polar axis z. Given a for example interlaminary surgical operation on the spinal column of a patient lying on his stomach, the endoscopy longitudinal axis, thus the polar axis preferably runs vertically. The hollow needle guides can be arranged meridionally in rows and each extend radially to the viewing region at a polar angle which is individual to each hollow needle guide. Preferably, the hollow needle guides extend as precisely as possible onto the middle point or onto an inner region of the viewing region. However, in principle it is sufficient if a hollow needle which is led in the respective hollow needle guide hits the viewing region with such an accuracy, that the distal tip of the hollow needle can be observed with an endoscope of the endoscopy unit.

The targeting device thus by way of the hollow needle guides unambiguously ensures that the distal tip of a hollow needle, irrespective of which of the hollow needle guides which are available is selected, always lands in the viewing region of the endoscope and can be finally positioned there whilst being viewed by way of the endoscope. For this reason, one can make do without an X-ray control and/or ultrasound control for placing the hollow needle, which significantly simplifies the surgical operation. The risk of an incorrect piercing is reduced by way of the hollow needle guides, since apart from the selection of the hollow needle guide it is only the piercing depth of the hollow needle and the azimuthal rotary position of the targeting device which remain as degrees of freedom. The azimuthal rotary position of the targeting device typically runs transversely to the (given a patient lying on his stomach, horizontally running) longitudinal axis of the spinal column, so that one pierces between the vertebral bodies in a selective manner obliquely from the left or obliquely from the right. Preferably, the targeting device by way of a defined stop on each hollow needle guide also determines the piercing depth of the hollow needle with a defined length, so that it is then the selection of the hollow needle guide which remains as the only degree of freedom once the azimuthal rotary position of the targeting device is selected. The selection of the hollow needle guide determines the polar angle of the piercing with respect to the longitudinal axis of the endoscope.

After a final placement of the guide wire which is situated in the hollow needle, the hollow needle can be pulled proximally out of the patient and the targeting device, wherein the guide wire remains positioned in the patient or is herein manually fixedly held in a guide wire section which projects proximally out of the hollow needle, by the operating person. The guide wire is preferably very much longer than the hollow needle. Due to the fact that each of the hollow needle guides is open to one of its two azimuthal sides over its entire length, the guide wire can be simply removed laterally from the targeting device and/or the targeting device can be pivoted azimuthally such that the guide wire slips laterally out of the hollow needle guide. The relatively long guide wire does not therefore need to be pulled distally out of the hollow needle amid great curvature and with large effort. The feature that each of the hollow needle guides is open to one of its two azimuthal sides over its entire length is therefore to be understood in that a guide wire which is situated in a hollow needle guide can be removed laterally out of the hollow needle guide after the hollow needle has been pulled proximally out of the hollow needle guide. In order to provide a guidance of the hollow needle, the hollow needle guides are not open to such an extent that the hollow needle itself can be removed laterally. The hollow needle can only be pulled out of the hollow needle guides proximally. Preferably, each of the hollow needle guides can have an inner diameter for axially receiving the hollow needle and at its open azimuthal side can comprise an opening slot with a clear slot width, wherein the clear slot width is smaller than the inner diameter of the hollow needle guides. The clear slot width is therefore preferably smaller than the outer diameter of the hollow needle, but larger than the outer diameter of a guide wire which is located in the hollow needle. Preferably, the clear slot width is smaller than the outer diameter of the hollow needle, but at least as large as the inner diameter of the hollow needle.

After the placing of the guide wire by way of the hollow needle has been effected and after the proximal withdrawal of the hollow needle from the selected hollow needle guide, the targeting device is firstly no longer needed and can be pivoted away azimuthally and/or be disassembled from the endoscopy unit. A rigid or flexible working sleeve can then be pushed distally over the guide wire into the patient in a known way and manner. Several working sleeves can then also be successively pushed over one another in order to achieve a necessary access cross section. As soon as the necessary access cross section is reached, the guide wire as well as possibly inner working sleeves can be pulled proximally out of the outermost working sleeve.

Optionally, each hollow needle guide can comprise a proximal guide end and a distal guide end, wherein the proximal guide ends of the hollow needle guides essentially have the same distance to the viewing region. Herewith, each hollow needle guide is available for the hollow needle with a defined length. Alternatively to this, for each hollow needle guide or groups of hollow needle guides, an associated length of the hollow needles which are to be used therein can be given. However, it is preferable for each hollow needle guide to be designed to be able to be used with a hollow needle of a defined length, i.e. the proximal guide ends of the hollow needle guides are arranged around the viewing region on a virtual spherical surface, wherein the virtual spherical surface has a radius which corresponds to the length of the hollow needle. The targeting device is therefore preferably designed in a manner adapted to a hollow needle length.

Optionally, for every pair which is arbitrarily selected from the hollow needle guides, it can be the case that the distal guide end of a first hollow needle guide of the pair extends along a first polar angle and has a first distance to the viewing region, and the distal guide end of a second hollow needle guide of the pair extends along a second polar angle and has a second distance to the viewing region, wherein the first polar angle is smaller than the second polar angle and the first distance is smaller than the second distance. The hollow needle guides with a greater distance to the endoscope longitudinal axis can be designed shorter than hollow needle guides with a smaller distance to the endoscope longitudinal axis. This makes particular sense if the endoscope longitudinal axis is aligned vertically from the above onto a spine of a patient lying on his stomach in the case for example of an interlaminary surgical operation, in order to keep a tilting moment which is exerted by the weight of the targeting device and the hollow needle as small as possible. The weight of the targeting device is therefore to be kept as slow as possible in the regions which are remote from the endoscope longitudinal axis.

Optionally, the proximal guide ends of the hollow needle guides can widen proximally in a funnel-like manner and each form a defined stop for the hollow needle. This is particularly expedient, in order to make it easier for an operating person to insert the hollow needle into the targeting device and to provide a visible examination of the correctly completely inserted hollow needle.

Optionally, adjacent hollow needle guides can be connected to one another each at the proximal guide end and at the distal guide end. A connection therebetween is not necessary and can be reduced by way of omitting the weight of the targeting device.

Optionally, the hollow needle guides can be open towards different ones of their two azimuthal sides in an alternating manner. This means that each second hollow needle guide is open to the right and the hollow needle guides which lie therebetween are open to the left, or vice versa. This has advantages in manufacture since the targeting device can then be manufactured of one piece in a simpler manner without material stresses which distort to the right or left.

Optionally, the targeting device further comprises a fastening element for the tool-free releasable fastening to the endoscopy unit. Preferably, the fastening element permits a secure, fixed connection which completely fixes the translatory positioning and rotational alignment of the targeting device relative to the endoscopy unit and secures it in this.

Optionally, the fastening element can be designed to at least partly embrace an access tube of the endoscopy unit in a designated manner and to be clamped onto this. An endoscope can then be inserted into the access tube independently of the targeting device. For this, the targeting device has a distance to the endoscope longitudinal axis proximally of the fastening element, so that an endoscope can be completely inserted into the access tube.

Optionally, the fastening element can be arranged at a distal end region of the targeting device. By way of this, the targeting device has an adequately large distance to the viewing region, so that the hollow needle guides can be arranged at an adequate distance to one another, in order with the associated polar angles to provide as many as possible different selection possibilities for the piercing angle over a large as possible polar angle region.

Optionally, the fastening element can comprise a quick release lever or clamping lever. Preferably, the fastening element can comprise a positive-fit element which is designed to form a positive fit with a corresponding positive-fit element which is provided on the endoscopy unit at a defined position along the endoscope longitudinal axis. On account of the positive fit of the positive-fit element at the defined position, the translatory positioning and rotational alignment of the targeting device relative to the endoscopy unit can be unambiguously defined, so that an incorrect assembly of the targeting device on the endoscopy unit can be ruled out. Thus preferably there is no degree of freedom in deviating from a correct assembly of the target device on the endoscopy unit for the assembly of the targeting device on the endoscopy unit.

Optionally, the hollow needle guides can be formed by a single-piece guide element. This makes sense in order to reduce the variety of parts of the targeting device and to avoid an inaccurate assembly of individual parts. The strict tolerance demands for the manufacture of hollow needle guides are easier to achieve with a single-piece guide element.

Optionally, the hollow needle guides can be additively manufactured as a single-piece unit. The entirety of the hollow needle guides can herein form the guide element which is manufactured as one piece. The total weight of the targeting device can be greatly reduced by way of an additive manufacture, for example by way of selective laser fusion (LPBF, laser powder bed fusion). The guide element which is manufactured of one piece and which forms the hollow needle guides can be manufactured additively from a lightweight metal, from a lightweight metal alloy and/or from a lightweight plastic. The material of the guide element can comprise open and/or closed cavities, in order to reduce the weight and to largely maintain the structural stability. For example, the total weight of the targeting device can be less than 100 grams, preferably 70-80 grams. The structural stability of the targeting device however in this case is characterized by a high stiffness.

Optionally, the targeting device can be azimuthally broken through between the hollow needle guides. In particular, this saves weight and material.

Optionally, at least one of the hollow needle guides can comprise a distal guide section and a proximal guide section, wherein the targeting device between the distal guide section and the proximal guide section comprises at least one free section, in which the hollow needle is not guided. This too can save weight and material, since the hollow needle basically only needs to be guided at two locations which are distanced as far as possible in the longitudinal direction, in order to unambiguously define the spatial alignment of the hollow needle.

Optionally, for each pair which is arbitrarily selected from the hollow needle guides, it can be that a first hollow needle guide of the pair extends along a first polar angle and a second hollow needle guide of the pair extends along a second polar angle, wherein the free section of the first hollow needle guide is longer than the free section of the second hollow needle guide and the first polar angle is smaller than the second polar angle. Optionally, for each pair which is arbitrarily selected from the hollow needle guides, it can be that a first hollow needle guide of the pair extends along a first polar angle and a second hollow needle guide of the pair extends along a second polar angle, wherein the length of the first hollow needle guide is larger than the length of the second hollow needle guide and the first polar angle is smaller than the second polar angle. The hollow needle guides can therefore be differently long, like the individual pipes of a panpipe, wherein the length reduces with an increasing polar angle. This reduces the tilting moment upon the endoscopy unit, said tilting moment being exerted by the weight of the targeting device.

Optionally, apart from the hollow needle guides, the targeting device can comprise open and/or closed hollow chambers, in order to save weight and material.

Optionally, the hollow needle guides can be arranged in a meridionally rowed manner. By way of this, the azimuthal extension of the targeting device is as narrow as possible. The meridional distance of the hollow needle guides amongst one another is as low as possible, in order to save material and weight, and is as large as possible, in order to be able to provide a sufficient selection of piercing angles over a large as possible polar angle region.

According to a further aspect of the present disclosure, an endoscopy system for an endoscopic-surgical operation is provided, wherein the endoscopy system comprises:
an endoscopy unit which extends essentially in an endoscopy longitudinal axis and defines a viewing region,
at least one hollow needle, and
a previously described targeting device for the targeted guidance of the at least one hollow needle towards the viewing region along a selectable polar angle with respect to the endoscope longitudinal axis, wherein the targeting device is fastened or can be fastened to the endoscopy unit.

Optionally, the endoscopy unit can comprise an access tube which extends essentially in the endoscopy longitudinal axis and an endoscope which can be inserted into the access tube from the proximal side.

Optionally, the targeting device can be releasably fastened to a proximal end section of the access tube in a tool-free manner.

Optionally, the hollow needle can be equipped with a guide wire which runs within the hollow needle, projects proximally out of the hollow needle and has an outer diameter which is smaller than the clear slot width of the azimuthal opening of each hollow needle guide of the targeting device.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1a is a schematic view of one surgical access possibility into an intervertebral space;

FIG. 1b is a schematic view of another surgical access possibility into an intervertebral space;

FIG. 1c is a schematic view of yet another surgical access possibility into an intervertebral space;

FIG. 2 is a lateral view of an embodiment example of an endoscopy system which is disclosed herein;

FIG. 3 is a lateral view upon the endoscopy system which is shown in FIG. 2, without an endoscope;

FIG. 4 is a lateral view onto a targeting device of the endoscopy system which is shown in FIG. 2;

FIG. 5a is a plan view upon the targeting device which is shown in FIG. 4;

FIG. 5b is view of a detail along longitudinal section A-A of FIG. 5a;

FIG. 6 is a plan view upon the endoscopy system which is shown in FIG. 3, without an endoscope and with a hollow needle which is withdrawn proximally out of the seventh hollow needle guide;

FIG. 7 is a perspective detailed view for the representation of how the targeting device which is shown in FIG. 4 is assembled on a distal end section of an access tube of the endoscopy system;

FIG. 8a is a detailed plan view before an assembly of the targeting device which is shown in FIG. 7, on a distal end section of an access tube of the endoscopy system;

FIG. 8b is a detailed plan view after an assembly of the targeting device which is shown in FIG. 7, on a distal end section of an access tube of the endoscopy system; and

FIG. 9 is a lateral view of a further embodiment example of an endoscopy system which is disclosed here, without an endoscope.

### DESCRIPTION OF PREFERRED EMBODIMENTS

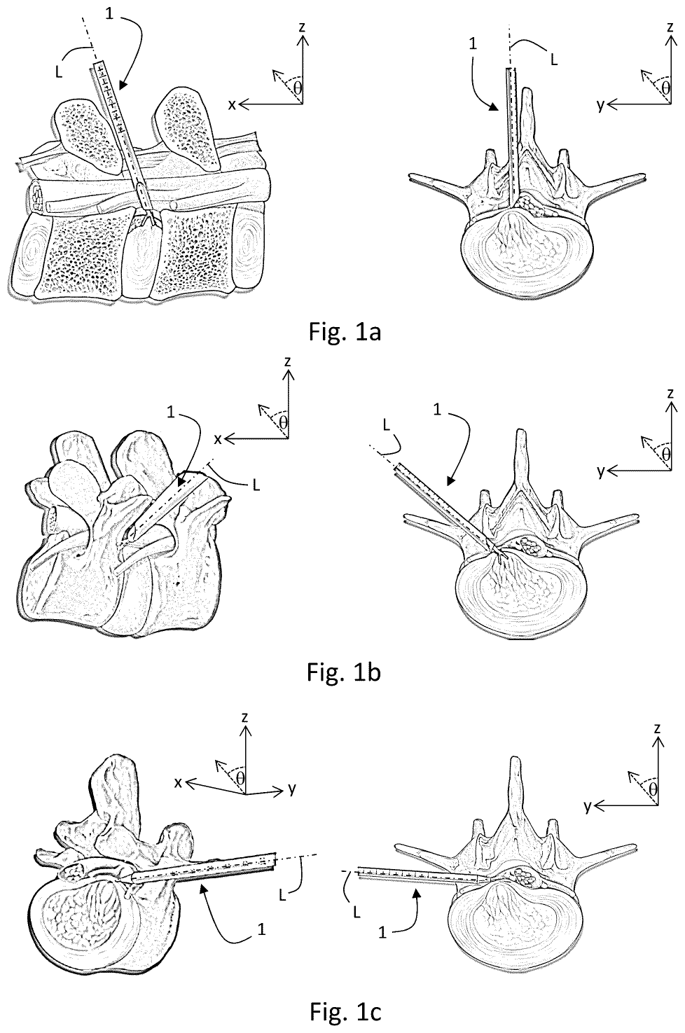

Referring to the drawings, three different surgical access possibilities into an intervertebral space are shown in FIG. 1a-c. What is to be seen in each case on the left is a longitudinal section or perspective section and on the right a cross section through a vertebra of a vertebral column of a patient lying on his stomach. For the simplification of the orientation, a right-handed Cartesian coordinate system is shown in all figures, concerning which the z-axis runs vertically, the x-axis horizontally along the vertebral column and the y-axis horizontally in a lateral manner. Furthermore, it is expedient to specify the relative spatial positions and alignments by way of spherical coordinates, wherein the polar angle θ here is defined as the angle to the z-axis, an azimuth angle φ as the rotary angle in the horizontal xy-plane and r as the distance to the coordinate origin.

An interlaminary access through which one surgically engages into an intervertebral space by way of an endoscopy unit 1 is shown in FIG. 1a. Only a distal end section of the endoscopy unit 1 is to be seen in FIGS. 1a-c. The endoscopy unit 1 defines an endoscopy longitudinal axis L which in the case of the interlaminary access which is shown in FIG. 1a runs in a relative vertical manner. FIG. 1b shows a transforaminal or extraforaminal access from the posterolateral side and FIG. 1c a transforaminal or extraforaminal access from the lateral side. Here it is to be noted that the patient can be placed such that the endoscopy unit 1 runs essentially vertically along the z-axis. If the patient remains lying on his stomach, it is expedient for the following figures to define the arbitrarily defined coordinate system such that the z-axis runs along the endoscopy longitudinal axis L.

For certain surgical operations, it is indeed advantageous or even necessary, additionally to the access which is created into the intervertebral space by way of the endoscopy unit 1, to create a second access into the intervertebral space from another direction.

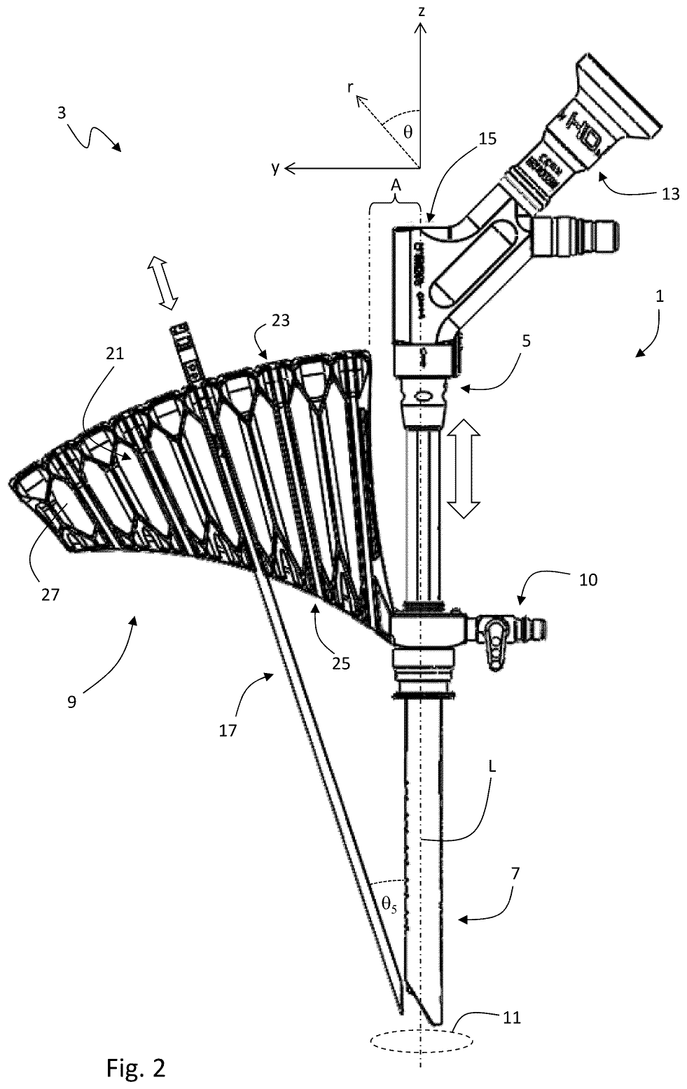

FIGS. 2 to 4 show an embodiment example of an endoscopy system 3 according to the invention which greatly simplifies the creation of a second access into the intervertebral space. The endoscopy system 3 comprises a conventional endoscopy unit 1, consisting of an endoscope 5 and an access tube 7, as well as a targeting device 9 and a hollow needle 17. The endoscopy unit 1 with the longitudinal axis L of the access tube 7 defines the endoscopy longitudinal axis L which here runs along the z-axis. The endoscope 5 can be distally introduced into the access tube 7 from the proximal side. The access tube 7 at its proximal end section comprises a rinsing fluid inlet and/or outlet 10. Once the endoscope 5 has assumed the maximal distal position in the access tube 7, the distal tip of the endoscope 5 is situated at the distal end of the access tube 7 for viewing a viewing region 11 which lies distally in front of the distal tip of the endoscope 5. The endoscope 5 preferably comprises an optical system for illuminating and viewing the viewing region 11. An operating person with his eye can directly view the viewing region 11 by way of an eyepiece 13 or (not shown) by way of an adapter can connect a camera onto the eyepiece 13 for imaging on a screen. The endoscope 5 preferably further comprises a working channel with a proximal working channel opening 15, through which an endoscopic tool such as for instance a resection forceps or scissors can be pushed to into the viewing region 11. By way of this, one can surgically operate amid viewing control of the intervertebral space.

The viewing region 11 here is represented in an oval manner and can be defined as a focusing plane which runs transversely to the endoscope longitudinal axis L, in the object space which lies distally in front of the endoscopy unit 1 and which can be observed by using the endoscope 5. A local spherical coordinate system of the endoscopy system 3 can therefore be defined such that the center of the spherical coordinate system is herein the center of the viewing region 11 which is arranged at the distal side in front of the endoscopy unit 1. The endoscopy longitudinal axis L is accordingly defined as a polar axis z. With regard for example to an interlaminary surgical operation on the spinal column of a patient lying on his stomach, the endoscopy longitudinal axis L, thus the polar axis z preferably runs vertically.

The targeting device 9 according to the invention serves for simplifying the creation of a second access to the viewing region 11 by way of the hollow needle 17. The targeting device 9 comprises a fastening element 19, here with a quick release lever 33 for the tool-free releasable fastening to a proximal end section of the access tube 7. The targeting device 9 is therefore fixedly locked on the access tube 7 during its application. As soon as the targeting device is no longer required after a successful placing of the hollow needle 17, it can be quickly disassembled in a tool-free manner, so that it does not get in the way given the continued surgical operation.

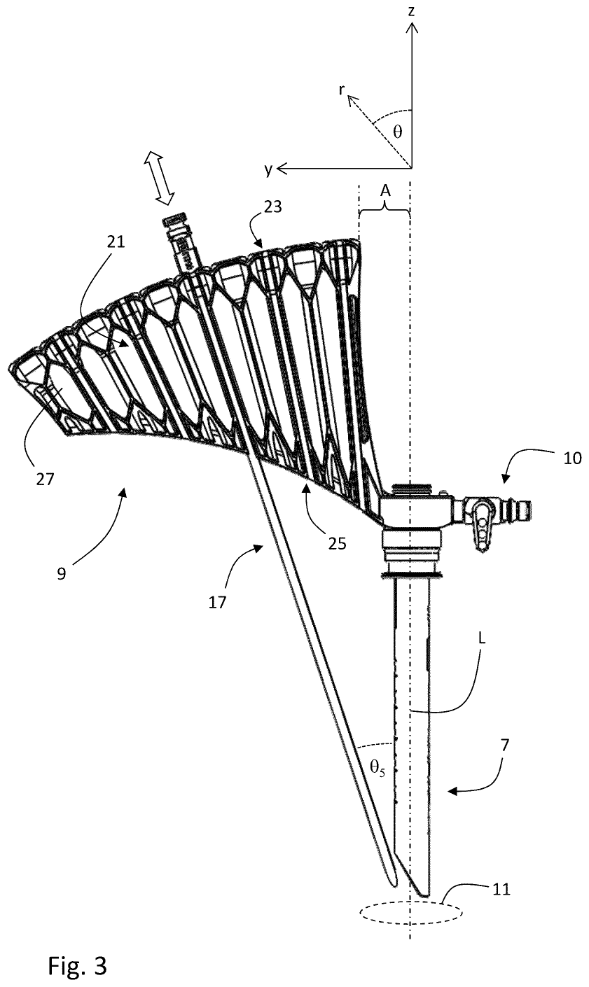

The targeting device 9 defines a plurality (here ten) of hollow needle guides 21 which are arranged next to one another in the manner of a fan and each extend towards the viewing region 11 along a different polar angle θ with respect to the endoscope longitudinal axis L. The hollow needle guides 21 are arranged rowed in a meridional manner and each extend radially towards the viewing region 11 at a polar angle θ which is assigned to each hollow needle guide 21. In FIGS. 2 and 3, the hollow needle 17 is inserted into the fifth hollow needle guide 21 which extends towards the viewing region 11 at a polar angle θ₅ with respect to the endoscope longitudinal axis L.

Each hollow needle guide 21 comprises a proximal guide end 23 and a distal guide end 25, wherein the proximal guide ends 23 of the hollow needle guides 21 essentially have the same distance to the viewing region 11. The proximal guide ends 23 of the hollow needle guides 21 are therefore arranged in a circle around the viewing region 11 in the yz-plane which is shown in FIGS. 2 to 4. The radius of the circle is determined by the length of the hollow needle 17. Herewith, each hollow needle guide 21 is available for the hollow needle 17 with a defined length, i.e. the targeting device 9 is designed in a manner adapted for a certain length of the hollow needle 17.

The hollow needle guides 21 with a greater distance to the endoscope longitudinal axis L are designed shorter than hollow needle guides with a smaller distance to the endoscope longitudinal axis L. Accordingly, the distance of the distal guide end 25 to the viewing region 11 is greater with hollow needle guides 21 with a greater distance to the endoscope longitudinal axis L than with hollow needle guides 21 with a smaller distance to the endoscope longitudinal axis L. This is particularly expedient for the case in which the endoscope longitudinal axis L for example with an interlaminary surgical operation is aligned vertically from above onto a vertebral column of a patient lying on his stomach, in order to keep a tilting moment which is exerted by the weight of the targeting device 9 as low as possible. The weight of the targeting device 9 is therefore as low as possible in particular in the regions which are remote from the endoscope longitudinal axis L. As is shown in FIG. 2, the targeting device 9 is preferably aligned diametrically to the alignment of the eyepiece 13 of the endoscope 5 in the circumferential direction about the endoscope longitudinal axis L, i.e. an azimuth angle φ of approximately 180° lies between these. By way of this, the tilting moments which are caused by the eye-piece 13 and the targeting device 9 at least partly cancel one another.

The proximal guide ends 23 of the hollow needle guides 17 widen proximally in a funnel-like manner and each form a defined stop for the hollow needle 17. This is particularly expedient in order to simplify the insertion of the hollow needle 17 into the targeting device 9 by an operating person and to provide a visual examination of the correctly completely inserted hollow needle 17. Herewith, the hollow needle 17 cannot be inadvertently pierced too deeply and the distal tip of the hollow needle 17 always lands in the viewed region 11, where it can be finally placed amid viewing control.

Concerning the targeting device 9, adjacent hollow needle guides 21 are connected to one another each at the proximal guide end 23 and the distal guide end 25. A connection therebetween is not necessary, so that the weight of the targeting device 9 is reduced there by way of omitting material. The targeting device 9 therefore comprises azimuthal openings 27 between the hollow needle guides 21.

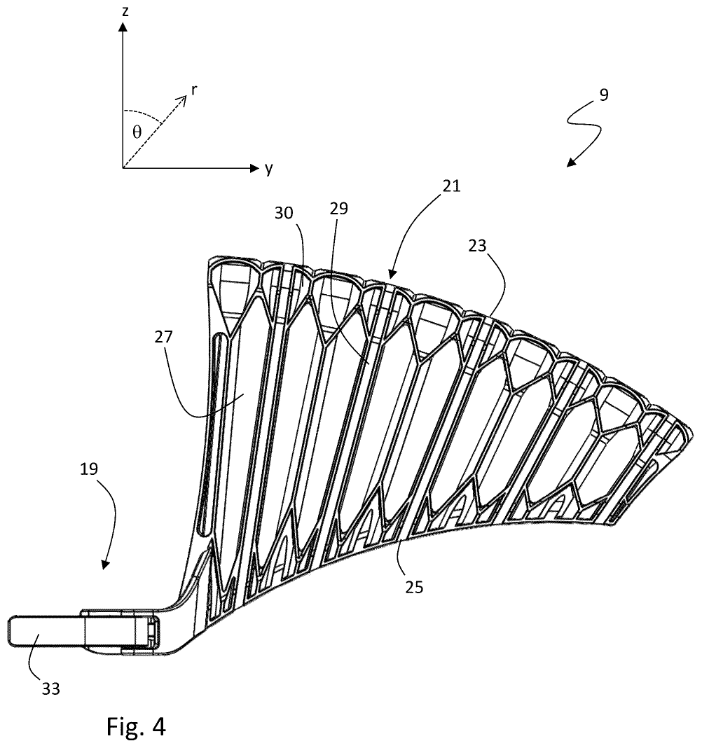

According to the invention, each of the hollow needle guides 21 is open towards one of its two azimuthal sides over its entire length. In the embodiment example which is shown in FIGS. 2 to 4, the hollow needle guides 21 are open towards different ones of their two azimuthal sides in an alternating manner. This means that every second hollow needle guide is open to the right and the intermediately lying hollow needle guides are open to the left or vice versa. In FIGS. 2 and 3, the first, third, fifth, seventh and ninth hollow needle guide 21 from the endoscope longitudinal axis L are open towards the viewer (negative x-direction), whereas the second, fourth, sixth, eighth and tenth hollow needle guide 21 from the endoscope longitudinal axis L are open away from the viewer (negative x-direction). The target device 9 is shown from the other azimuthal side in FIG. 4. The design which is open to the left and right in an alternating manner has advantages in manufacture, since the targeting device 9 is then easier to manufacture of one piece without having distortions to the right or left.

The feature that each of the hollow needle guides 21 is open towards one of its two azimuthal sides over its entire length is thus to be understood such that a guide wire (not shown) which is located in a hollow needle guide 21 can be pulled laterally out of the hollow needle guide 21 after the hollow needle 17 has been pulled proximally out of the hollow needle guide 21. In order to provide a guidance of the hollow needle 17, the hollow needle guides 21 are not open to such an extent that the hollow needle 17 itself can be removed laterally. The hollow needle 17 can only be pulled out of the hollow needle guides 21 proximally.

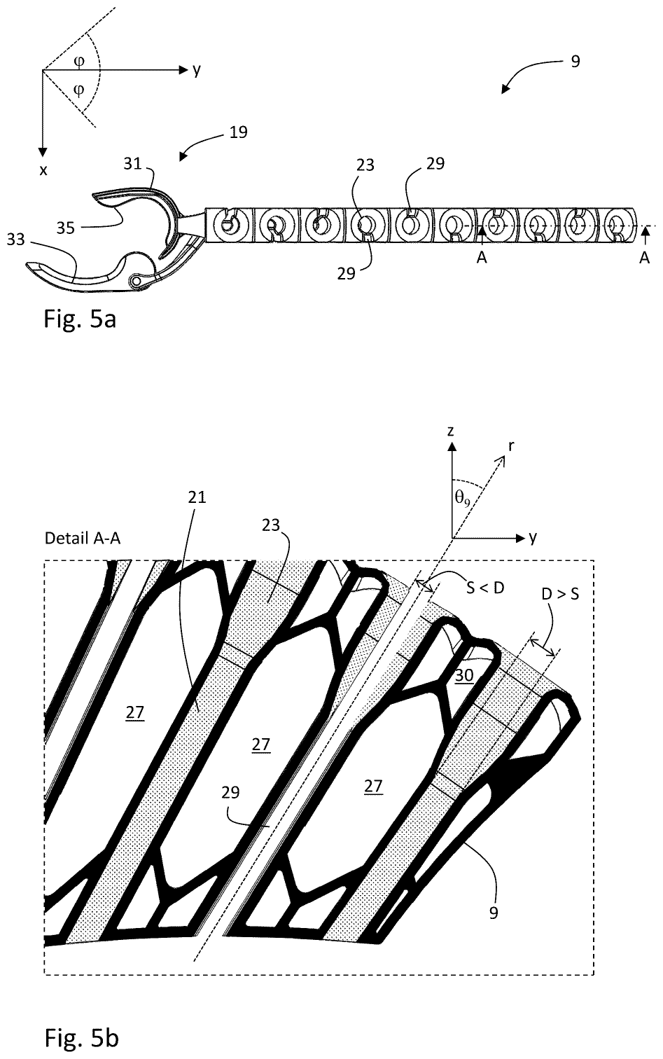

As is shown in more detail in FIGS. 5a,b, each of the hollow needle guides 21 has an inner diameter D for axially receiving the hollow needle 17. The inner diameter D is the same for all hollow needle guides 21, so that each can receive the hollow needle 17 with the respective outer diameter in an exactly fitting manner. Each hollow needle guide 21 at its open azimuthal side comprises an opening slot 29 with a clear slot width S, wherein the clear slot width S is smaller than the inner diameter D of the hollow needle guides. The clear slot width S is therefore smaller than the outer diameter of the hollow needle 17, but larger than the outer diameter of a guide wire (not shown) which is located in the hollow needle 17. Preferably, the clear slot width S is smaller than the outer diameter of the hollow needle 17, but at least as large as the inner diameter of the hollow needle 17.

For reducing the variety of parts, the costs and the weight of the targeting device 9, the hollow needle guides 21 are formed by a single-piece guide element. The strict tolerance demands for the manufacture of the hollow needle guides 21 are furthermore easier to achieve by way of a single-piece guide element. The single-piece guide element here is additively manufactured. The entirety of the hollow needle guides 21 herein forms the guide element which is manufactured as one piece. The total weight of the targeting device 9 can be greatly reduced by way of an additive manufacture, for example by way of selective laser powder bed fusion (LPBF). For example, the total weight of the targeting device 9 can be less than 100 grams, preferably 70 to 80 grams. The structural stability of the targeting device 9 however is characterized at all events by a high stiffness. Apart from the hollow needle guides 21, the targeting device 9 can comprise open and/or closed hollow chambers 30, in order to save weight and material.

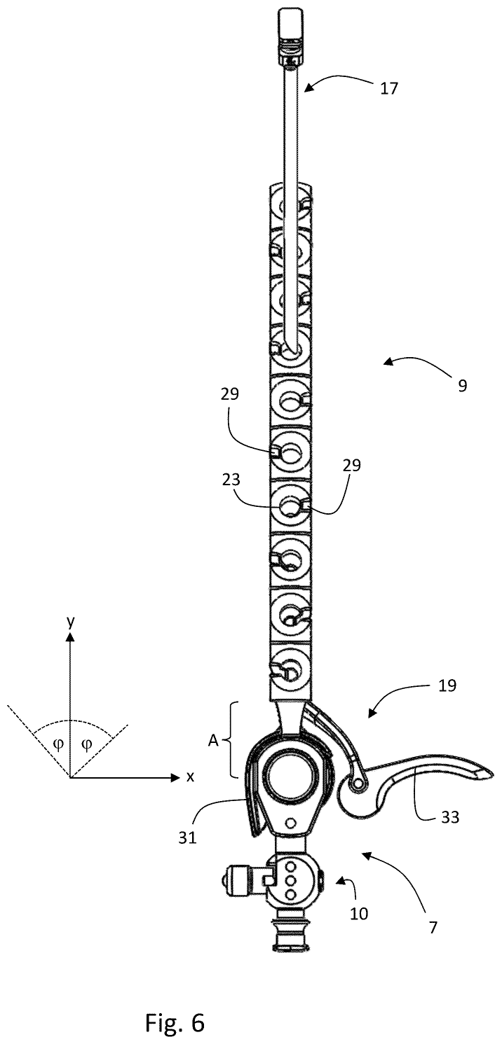

In a plan view which is shown in FIG. 6, the hollow needle 17 is shown just before the insertion into the hollow needle guide 21 which is seventh from the endoscopy longitudinal axis L. The fastening element 19 is designed to at least partly embrace the access tube 7 of the endoscopy unit 1 in a designated manner and to be clamped thereon. The endoscope 5 can then be inserted into the access tube 7 independently of the locked targeting device 9. For this, the targeting device 7 proximally of the fastening element 19 has a distance A to the endoscope longitudinal axis L, so that the endoscope 5 can be completely inserted into the access tube 7.

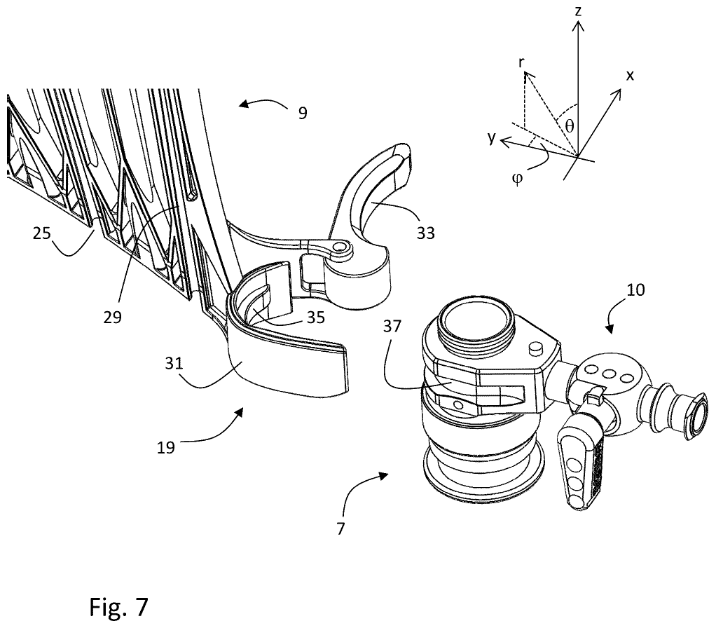

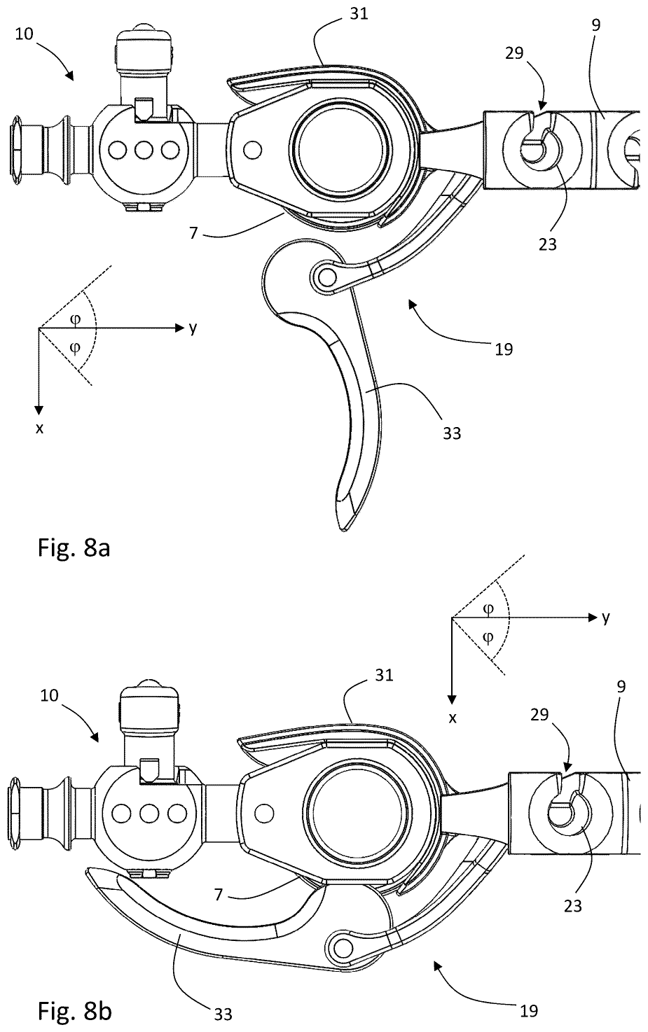

The fastening element 19 which is shown more precisely in FIG. 7 and FIGS. 8a,b here comprises a quick release lever 33 lying azimuthally opposite a grip hook 31 of the targeting device 9 which embraces the proximal end section of the access tube 7. The grip hook 31 here comprises a positive-fit element 35 which is adapted to the proximal end section of the access tube 7, in the form of a web which runs at the inner side in the circumferential direction, in order to form a positive-fit with a correspondingly shaped positive-fit element 37 in the form of a groove on the access tube 7, said groove running on the outer side in the circumferential direction. Due to the positive fit of the positive-fit elements 35, 37 with one another at the defined position, the translatory positioning and rotatory alignment of the targeting device relative to the endoscopy unit is unambiguously defined, so that an incorrect assembly of the targeting device 9 on the endoscopy unit 1 can be ruled out. Thus for the assembly of the targeting device 9 on the endoscopy unit 1 there is no degree of freedom in deviating from a correct assembly of the targeting device 9 on the endoscopy unit 1.

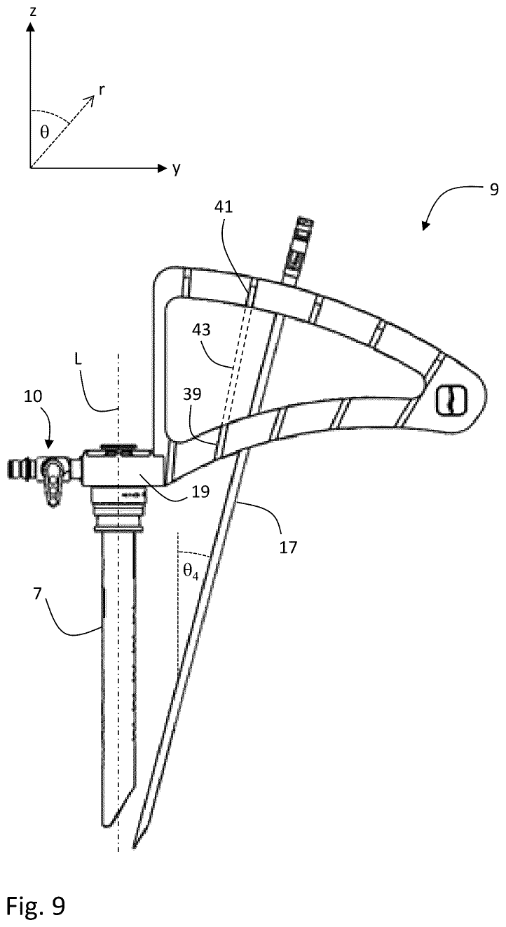

FIG. 9 shows a different embodiment example of a targeting device 9 which is fastened to the access tube 7. The less complex structure of the targeting device 9 as the case may be can be more simply manufactured in a non-additive method, for example molded and/or milled. The in total nine hollow needle guides 21 here comprise a distal guide section 39 and a proximal guide section 41, wherein the targeting device 9 comprises at least one free section 43 between the distal guide section 39 and the proximal guide section 41, in which free section the hollow needle 17 is not guided. This can also save weight and material since the hollow needle 17 in principle only needs to be guided at two locations which are distanced as far as possible in the longitudinal direction, in order to unambiguously define the spatial alignment of the hollow needle 17. The length of the free section 43 is longer for hollow needle guides 21 which are situated more closely to the endoscopy longitudinal axis L than for hollow needle guides 21 which are distanced further to the endoscopy longitudinal axis L. The characteristics of the embodiment examples according to FIGS. 2 to 8a,b can be arbitrarily combined with the characteristics of the embodiment example according to FIG. 9. In FIG. 9, the hollow needle 17 is inserted into the fourth hollow needle guide 21 which extends along the polar angle θ₄ relative to the endoscopy longitudinal axis L and as the second, sixth and eighth hollow needle guide 21 is open to the non-visible azimuthal side. The first, third, fifth, seventh and ninth hollow needle guide 21 are each open to the visible azimuthal side.

While specific embodiments of the invention have been shown and described in detail to illustrate the application of the principles of the invention, it will be understood that the invention may be embodied otherwise without departing from such principles.

### List of Reference Numerals

1 endoscopy unit
3 endoscopy system
5 endoscope
7 access tube
9 targeting device
10 rinsing fluid inlet and/or outlet
11 viewing region
13 eyepiece
15 proximal working channel opening
17 hollow needle
19 fastening element
21 hollow needle guide
23 proximal guide end
25 distal guide end
27 opening
29 opening slot
30 hollow chamber
31 grip hook
33 quick release lever
35 positive-fit element
37 positive-fit element
39 distal guide section
41 proximal guide section
43 free section
A distance
D inner diameter
S clear slot width
θ polar angle
φ azimuth angle

1. A targeting device for a targeted guidance of a hollow needle with an endoscopic-surgical operation, wherein the targeting device is fastened or can be fastened to an endoscopy unit which extends essentially in an endoscopy longitudinal axis and defines a viewing region, wherein the targeting device defines a plurality of hollow needle guides, wherein each of the hollow needle guides each extends towards the viewing region along a different polar angle with respect to the endoscopy longitudinal axis, wherein each of the hollow needle guides is open towards one of its two azimuthal sides over its entire length.

2. A targeting device according to claim 1, wherein each hollow needle guide comprises a proximal guide end and a distal guide end, wherein the proximal guide end of each of the hollow needle guides essentially have the same distance to the viewing region.

3. A targeting device according to claim 2, wherein for every pair which is selected from the hollow needle guides, the distal guide end of a first hollow needle guide of a pair extends along a first polar angle and has a first distance to the viewing region and the distal guide end of a second hollow needle guide of the pair extends along a second polar angle and has a second distance to the viewing region, wherein the first polar angle is smaller than the second polar angle and the first distance is smaller than the second distance.

4. A targeting device according to claim 2, wherein the proximal guide end of each of the hollow needle guides widen proximally in a funnel-like manner and each forms a defined stop for the hollow needle.

5. A targeting device according to claim 2, wherein adjacent hollow needle guides are connected to one another each at the proximal guide end and at the distal guide end.

6. A targeting device according to claim 1, wherein the hollow needle guides are open towards different ones of their two azimuthal sides in an alternating manner.

7. A targeting device according to claim 1, further comprising a fastening element for a tool-free releasable fastening to the endoscopy unit.

8. A targeting device according to claim 7, wherein the fastening element is configured to at least partly embrace an access tube of the endoscopy unit in a designated manner and to be clamped onto the access tube.

9. A targeting device according to claim 7, wherein the fastening element is arranged at a distal end region of the targeting device.

10. A targeting device according to claim 7, wherein the fastening element comprises a quick release lever or clamping lever.

11. A targeting device according to claim 7, wherein the fastening element comprises a positive-fit element configured to form a positive fit with a corresponding positive-fit element provided on the endoscopy unit at a defined position along the endoscopy longitudinal axis.

12. A targeting device according to claim 1, wherein the hollow needle guides are formed by a single-piece guide element.

13. A targeting device according to claim 1, wherein the hollow needle guides are additively manufactured as a single-piece unit.

14. A targeting device according to claim 1, wherein the targeting device is azimuthally broken through between the hollow needle guides.

15. A targeting device according to one of the preceding claim 1, wherein at least one of the hollow needle guides comprises a distal guide section and a proximal guide section, wherein the targeting device between the distal guide section and the proximal guide section comprises at least one free section, in which the hollow needle is not guided.

16. A targeting device according to claim 15, wherein for each pair selected from the hollow needle guides, a first hollow needle guide of a pair extends along a first polar angle and a second hollow needle guide of the pair extends along a second polar angle, wherein the at least one free section of the first hollow needle guide is longer than the at least one free section of the second hollow needle guide and the first polar angle is smaller than the second polar angle.

17. A targeting device according to claim 1, wherein for each pair selected from the hollow needle guides, a first hollow needle guide of a pair extends along a first polar angle and a second hollow needle guide of the pair extends along a second polar angle, wherein a length of the first hollow needle guide is larger than a length of the second hollow needle guide and the first polar angle is smaller than the second polar angle.

18. A targeting device according to claim 1, wherein each of the hollow needle guides has an inner diameter for axially receiving the hollow needle and at its open azimuthal side comprise an opening slot with a clear slot width, wherein the clear slot width is smaller than the inner diameter.

19. A targeting device according to claim 1, wherein apart from the hollow needle guides, the targeting device comprises open and/or closed hollow chambers.

20. A targeting device according to claim 1, wherein the hollow needle guides are arranged in a meridionally rowed manner.

21. An endoscopy system for an endoscopic-surgical operation, the endoscopy system comprising:
an endoscopy unit extending essentially in an endoscopy longitudinal axis [[(L)]] and defines defining a viewing region;
at least one hollow needle; and
a targeting device for a targeted guidance of the at least one hollow needle towards the viewing region along a selectable polar angle with respect to the endoscopy longitudinal axis, wherein the targeting device is fastened or can be fastened to the endoscopy unit, wherein the targeting device defines a plurality of hollow needle guides, wherein each of the hollow needle guides extends towards the viewing region along a different polar angle with respect to the endoscopy longitudinal axis, wherein each of the hollow needle guides is open towards one of its two azimuthal sides over its entire length.

22. An endoscopy system according to claim 21, wherein the endoscopy unit comprises an access tube extending essentially in the endoscopy longitudinal axis and an endoscope which can be inserted into the access tube from a proximal side.

23. An endoscopy system according to claim 22, wherein the targeting device is releasably fastened to a proximal end section of the access tube in a tool-free manner.
